(19) United States
(12) Patent Application Publication
(10) Pub. No.: US 2016/0206472 A1
(43) Pub. Date: Jul. 21, 2016

(54) CONTROL DEVICE FOR A LASSER SYSTEM AND LASER SYSTEM FOR CONTROLLING THE LASER SYSTEM

(71) Applicant: CARL ZEISS MEDITEC AG, Jena (DE)

(72) Inventor: Martin Kraus, Huettlingen (DE)

(21) Appl. No.: 15/025,578

(22) PCT Filed: Sep. 30, 2014
(86) PCT No.: PCT/EP2014/002662

§ 371 Date: Mar. 29, 2016

(30) Foreign Application Priority Data
Sep. 30, 2013 (DE) 10 2013 016 335.8

(51) Int. Cl. A61F 9/008

(52) U.S. Cl. CPC A61F 9/008; A61F 2009/00897; A61B 34/20

(57) ABSTRACT

A control device for a laser system with a laser for introducing a cut into tissue of a human or animal eye, wherein the control device is set up to arrange and move a focal point of a laser beam of the laser within the tissue. The control device includes an actuator configured to move the focal point along a continuous cutting path starting from a starting point arranged within the tissue between the starting point and an outer edge of the tissue in order to dissect the tissue into at least one continuous tissue strand.

### CROSS REFERENCE TO RELATED APPLICATIONS

This application is a U.S. National Stage Application under 35 U.S.C. §371 of International Application No. PCT/EP2014/002662 filed on Sep. 30, 2014, and claims benefit to German Patent Application No. DE 10 2013 016 335.8 filed on Sep. 30, 2013. The International Application was published in German on Apr. 2, 2015 as WO 2015/043772 A1 under PCT Article 21(2).

### FIELD

The invention relates to a control device for a laser system capable of introducing a cut into the tissue of a human or animal eye, to a laser system with such a control device, and to a method for controlling such a laser system.

### BACKGROUND

With many eye diseases it is necessary to remove the lens of the eye and replace it with an artificial lens. The lens is arranged in a capsular bag in the eye and is surrounded by a capsule membrane. In order to remove the lens, it must be separated from the capsular bag without damaging the capsular bag because the latter usually needs to be used to receive the artificial lens. For this, the lens is firstly roughly divided or cut up into several parts (pre-fragmentation), in particular using a laser which focuses high-energy, pulsed laser radiation into a section plane within the tissue of the lens.

The following steps are usually carried out to remove a lens. Firstly, a laser introduces one or more cuts or incisions and carries out a pre-fragmentation of the lens by means of a plurality of rough, individual cuts. The lens is cut up into several parts by means of the laser, e.g. divided by radially aligned cuts, like a cake. The capsule membrane which was previously cut out of the capsular bag by means of capsulorhexis can then be removed using an aspiration handpiece. A hydrodissection can also be effected, in particular when the lens can only be released from the capsule membrane with difficulty. The hydrodissection is a stand-alone process step. Using an ultrasonic handpiece, a further fine fragmentation of the individual pre-fragmented parts of the lens can then be effected (emulsification). Finally, the finely fragmented parts can be suctioned off using an aspiration handpiece. The aspiration handpiece is moved within the eye in order to be able to aspirate the individual parts. An operator must take care to guide the aspiration handpiece in such a way that the capsular bag is not damaged. Because of the intermediary ultrasonic fragmentation, it is necessary to insert an instrument into the eye several times. The time required for the treatment is e.g. in the range of from three to five minutes.

US 2009/0012507 A1 describes a method for fragmenting a lens of an eye in which, for the purpose of removing the lens, an optical segmentation is introduced using a spiral-shaped cut in a nucleus of the lens.

### SUMMARY

In an embodiment, the present invention provides a control device for a laser system with a laser for introducing a cut into tissue of a human or animal eye, wherein the control device is set up to arrange and move a focal point of a laser beam of the laser within the tissue. The control device includes an actuator configured to move the focal point along a continuous cutting path starting from a starting point arranged within the tissue between the starting point and an outer edge of the tissue in order to dissect the tissue into at least one continuous tissue strand.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 is a schematic representation of the structure of a control device according to an embodiment of the invention in conjunction with a laser system;

FIG. 2 is a schematic representation of a lateral sectional view of a human eye;

FIG. 3 is a schematic top view of a lens which is dissected along a spiral-shaped cutting path according to an embodiment of the invention;

FIG. 4 is a schematic top view of a lens which is dissected along a spiral-shaped cutting path according to a further embodiment of the invention;

FIG. 5a is a schematic representation of a lateral sectional view of a lens which is dissected along a spiral-shaped cutting path according to a further embodiment of the invention;

FIG. 5b is a schematic representation of a lateral sectional view of a variant of the embodiment example shown in FIG. 5a, namely a lens which is divided in several planes and in each plane is dissected along a spiral-shaped cutting path;

FIG. 6 is a lateral sectional view of a lens which is dissected along a spiral-shaped cutting path according to a further embodiment of the invention;

FIG. 7 is a schematic top view of a lens which is dissected along spiral-shaped cutting paths according to a further embodiment of the invention; and

FIG. 8 is a schematic top view of a lens which is dissected along a spiral-shaped cutting path according to a further embodiment of the invention.

### DETAILED DESCRIPTION

Embodiments of the present invention provide a device and a method whereby a laser can be controlled in such a way that tissue, in particular a lens, can be easily removed from an eye.

Embodiments of the invention provide a control device for a laser system with a laser, in particular a femtosecond laser, for introducing a cut into tissue of a human or animal eye, in particular into a lens of the eye, wherein the control device is set up to arrange and move a focal point of a laser beam of the laser within the tissue; wherein the control device is set up to move the focal point along a continuous cutting path extending two- or three-dimensionally, in particular in a single cut, starting from a starting point arranged within the tissue, in particular in the entire tissue, between the starting point and an outer edge of the tissue and to dissect the tissue into at least one continuous, in particular spiral-shaped tissue strand, in particular a tissue strand which extends from a central point or at least an approximately centrally arranged starting point to the edge of the tissue. The tissue strand preferably connects an outer surface of the lens to the lens nucleus. The entire tissue preferably corresponds to the entire lens. The laser can also cut the tissue strand three-dimensionally in all three spatial directions, in particular in the case of a relatively soft lens.

By guiding a laser along a continuous cutting path extending two- or three-dimensionally, the entire lens can be dissected without being divided into individual parts. This makes it possible to suction the lens out of the eye in one piece, as, metaphorically speaking, in the case e.g. of a spirally coiled rope which is pulled away by one end. The control device is preferably set up to dissect the lens in such a way that a single lens strand is formed which goes through the entire lens and preferably runs in all areas of the lens, in particular in relation to the radial extent of the lens. The control device is set up to dissect the entire lens into a continuous body which can be unrolled. An operator can hereby suction off the entire lens in one go. It is no longer necessary to suction off individual, small parts of the lens separately in each case. Also, the aspiration handpiece no longer needs to be guided near to the capsular bag.

An embodiment of the invention is based on the idea that it is advantageous if the lens can be subdivided in the radial direction in all areas, with the result that the radially outerlying areas of the lens can also be suctioned off starting from a central point of the lens. A lens strand which extends from the central point to the edge of the lens brings a range of advantages:
1. The operator no longer needs to guide the handpiece near to the capsular bag and can thus work under less stress because the integrity of the capsular bag is less vulnerable.
2. The operator needs to shift the handpiece less, which reduces the stress at the incision point and thus the post-operative astigmatism induced thereby.

The control device can be equipped with the following components:
1. A focus-measuring apparatus which is set up to determine a distance of a focal point of a pulsed laser beam of the laser in relation to a reference point of the laser; and
2. An optical capture unit which is set up to capture a position and/or geometry of the tissue relative to the reference point.

The geometry of the tissue or of the lens need not necessarily be captured in real time but can also be stored in a memory of the control device. The control device is preferably set up, provided e.g. the position of a front surface of the lens is known, to approximate the geometry or the size of the lens also on the basis of empirical values or of the age and the size of the person to be treated.

The control device is preferably set up to predetermine and calculate the cutting path. However, it is not necessary to calculate the cutting path in all cases. If e.g. both of a patient's eyes are to be treated, the geometry of one lens can be used to deduce the geometry of the other lens.

Further advantages result in the case of a tissue strand which can be suctioned out of the eye in one piece. The ultrasound energy introduced into the eye can be decreased, in particular reduced to about zero. An incision in the eye can thereby be made smaller and/or the diameter of an aspiration handpiece (or of an irrigation+aspiration handpiece) can be made larger, in particular because only an aspiration handpiece (or irrigation+aspiration handpiece) without an ultrasonic device has to be used. Hitherto, a conventional ultrasonic handpiece which can ensure both the aspiration and an irrigation usually had to be used. Preferably, suctioning-off took place through a central lumen in a tip of the ultrasonic handpiece. The irrigation is effected e.g. past the sides of the ultrasonic tip through a so-called “irrigation sleeve”, wherein the irrigation also serves to cool the ultrasonic tip. For physical reasons, the ultrasound requires a certain material strength at the tip, so that the latter does not break during operation. A handpiece with only irrigation/aspiration (without ultrasonic function) can therefore be thinner. A free end, i.e. the tip of the aspiration handpiece, also need not be moved within the eye, or at least only in a small area. The treatment can be carried out more easily and more quickly, expressed colloquially: “in one go”.

An ultrasound applicator for the ultrasonic fragmentation of individual parts of a lens can be dispensed with. In the ideal case, the entire lens can be removed solely by means of an aspiration handpiece without it being necessary to change instrument. The patient or the eye is spared by this, be it in respect of the input of ultrasound energy, in respect of the length of treatment, in respect of the stress or size of the incision in the eye or in respect of a massive reduction in the irrigation fluid rinsing volume, which is known to be directly related to the loss of endothelial cells during every cataract operation. In the ideal case, during the suctioning-off of the lens strand only as much irrigation fluid is fed into the eye as is lost through the volume of the suctioned-off lens.

A single cut can be arranged on the cutting path and the cut can be produced continuously by pulsed laser radiation. Many individual pulses can form a cutting line. The cut can also be formed by individual successive sections which also dissect the tissue, e.g. if the laser is not guided continuously but is to insert a stop at particular points, e.g. turning points.

The control device is preferably set up to lay the cutting path both within a nucleus of the lens and within a cortex of the lens. A dissection of both the nucleus and the cortex into a continuous tissue strand brings the advantage that the nucleus and the cortex can be removed together in one go or method step. Further preferably, the cutting path goes through the entire lens, starting from a starting point which is arranged at least approximately centrally (in relation to a longitudinal and/or transverse plane of the lens), to a surface or to a short distance from the surface of the lens.

The cutting path is preferably laid on circular tracks or on a spiral track around a central point in such a way that the tracks neighbouring each other in the radial direction have a distance from each other in the radial and/or axial direction (in each case) which at least approximately corresponds to the internal diameter of a lumen of an aspiration handpiece. It can hereby be ensured that the tissue can be suctioned off easily by the aspiration handpiece. Preferably, the distance (and thus the thickness or the diameter of a tissue strand) is not substantially larger than the internal diameter of the lumen, and further preferably the distance is, as far as possible, not smaller than the internal diameter of the lumen. Particularly preferably, the distance is slightly larger than the internal diameter. The tissue strand can hereby be more effectively suctioned off, since it has been shown that it is advantageous, due to the elasticity of the lens material, if the tissue strand rests against the lumen with a certain lateral pressure. The lateral pressure can improve the seal between the lumen and the tissue strand. By matching the distance to the internal diameter, the suction effect can be improved. The efficiency during the treatment can thereby be increased. As large as possible a diameter of the tissue strand increases the probability that the entire (lens) tissue, i.e. both the lens nucleus and the lens cortex, can be removed from the eye in one strand without the tissue strand tearing.

According to a variant, the distance (and thus the thickness or the diameter of a tissue strand) in an area of the lens nucleus is chosen to be somewhat smaller than the internal diameter of the lumen, since this is a harder tissue than in the area of the lens cortex, wherein the distance is enlarged increasingly along the cutting path. The distance from the end of the cutting path can at least approximately correspond to the internal diameter of the lumen, or also be somewhat larger. The control device is set up to lay the cutting path such that the cross section of the lens strand is continuously enlarged. This gradual thickening of the tissue strand towards the outside (and tapering towards the inside) brings the advantage that the lens tissue can be suctioned off with at least approximately the same resistance and thus the same speed. This is because the outerlying lens tissue (lens cortex) is usually much softer and more elastic than the lens nucleus, in particular a central part of the lens nucleus. The suctioning-off proceeds more quickly (more efficiently) when the lumen is completely filled, without producing too much friction there.

A gradual thickening of the tissue strand towards the outside can also bring an advantage when the tissue of the lens cortex tends to be more elastic than the tissue of the lens nucleus. This is because, during the aspiration of the lens strand, a tensile force is exerted on the lens strand in dependence on the negative pressure produced, which tensile force causes a pinching of the lens strand and thus a smaller cross-sectional area of the lens strand. Depending on the hardness of the lens tissue, and also in dependence on a (leakage) flow which may possibly be present between the lens strand and the internal surface of the lumen of the application handpiece it is more advantageous to keep the size of the cross-sectional area at least approximately constant or to enlarge it towards the outside.

The control device preferably has an arithmetic unit which is set up to calculate the cutting path, in particular in dependence on the geometry of the tissue. Alternatively, the cutting path can also be formed according to a predetermined cutting pattern starting from a starting point arranged centrally in the lens. The cutting pattern is preferably a spiral which extends outwards in the shape of a spiral starting from the centre of the lens, i.e. two- or three-dimensionally. In the case of a predetermined cutting pattern, the control device can be set up to form the cutting path only up to a radial distance from the centre of the lens, with the result that the cutting path does not run as far as a surface but remains arranged within the lens tissue.

Preferably, the starting point is arranged in a central point of the tissue. In the case of a lens, the central point can be determined, for example, by capturing the position of an outer circumference of the lens or the lens diameter and a thickness of the lens.

Preferably, the cutting path runs along a continuous curve. Further preferably, the curve has no kinks but has a particular minimum radius of curvature in all areas.

According to an embodiment, the control device is set up to capture the position of the outer edge of the tissue and to arrange the cutting path at least in sections at a predeterminable minimum distance from the outer edge. The cutting path can hereby be arranged at least in sections at a predeterminable minimum distance from an outer edge, in particular a surface, of the tissue. The minimum distance can be maintained except e.g. for a single access cut or access point. The position of the outer edge can be captured by means of an optical capture unit of the control device. In other words, the control device is set up to control the laser to move the focal point according to a continuous cutting path into an edge area of the tissue, wherein the cutting path can remain arranged at a minimum distance from the edge area. It is hereby possible to avoid, or to reduce the risk of, damage to the capsular bag receiving the lens. A protection mechanism can be provided for the capsular bag. Preferably, the cutting path runs parallel to the surface at least in sections in the area of the surface, in particular at a distance which at least approximately corresponds to the internal diameter of a lumen of an aspiration handpiece.

According to a variant, it can be advantageous not to cut the tissue strand into the lens right up to the capsular bag but to leave an uncut lens edge zone between the capsular bag and the end of the cutting path, which zone is easily torn away from the capsule in a subsequent suction step. Preferably, the control apparatus is set up to leave an uncut lens edge zone at the outer edge of the lens with a thickness which roughly corresponds to the thickness of the lens cortex, preferably 0.5 to 0.6 mm. It has been shown that the lens cortex can be removed comparatively easily.

According to an embodiment, the control device is set up to capture a central point of the tissue and to arrange the cutting path around the central point. Here, the cutting path can be arranged, starting from a starting point arranged centrally in the tissue, along a curve radially outwards in the direction of an outer edge, in particular a surface, of the tissue. In the case of a curve which runs radially outwards, the tissue strand can be formed such that it can be suctioned off safely and easily, in particular starting from the central starting point. By central starting point is preferably meant a geometric central point of the tissue, in particular the central point of the lens. The central point can be based on a surface of the tissue or also on the volume of the tissue.

According to a variant, the tissue strand has an at least approximately constant thickness in relation to a longitudinal section of the tissue strand, in particular in the area of the lens nucleus, which usually has a largely constant elasticity. This simplifies the suctioning-off with an aspiration handpiece, which has an internal lumen with a predetermined internal diameter. According to a variant, the cutting path runs along a continuous curve without kinks, the radius of curvature of which becomes larger monotonically as the distance from the starting point increases.

According to a variant, in particular in the case of a very hard lens nucleus, the lens is divided in an area between the lens nucleus and the lens cortex. Preferably, a division exclusive of the section of the tissue strand which connects the lens nucleus to the lens cortex is effected. In other words, the control device is set up to form a single tissue strand in the entire tissue and to divide the tissue strand into two successive tissue strand sections at the interface between the lens nucleus and the lens cortex. In this way, a distal end of an aspiration handpiece can also be applied at the interface between the lens nucleus and the lens cortex and at first only suction off the lens cortex, in particular if the lens nucleus is too hard to be suctioned off in one piece. The lens nucleus can then remain in the eye at first and be fragmented conventionally, in particular by means of ultrasound, in a further step.

According to an embodiment, the control device is set up to form the tissue strand with a constant thickness and/or a thickness which increases at least in sections. Here, the cutting path can be a spiral-shaped curve which can be arranged three-dimensionally around a starting point arranged centrally in the tissue in such a way that the tissue strand has a constant thickness at least in sections and/or has a thickness which increases at least in sections.

In the case of a lens, a continuous tissue body which is appropriate for the anatomical structure of the lens can be provided by means of a spiral-shaped cutting path. Metaphorically speaking, the lens is structured like an onion, as its consistency decreases towards the outside. A hard nucleus is surrounded by a relatively soft lens cortex. By means of a spiral-shaped dissection of the lens, a dissection can therefore be effected according to the different layers or degrees of hardness of the lens. This brings advantages for the suctioning-off in one piece intended by the one-piece dissection. The suctioning-off is started in the hard nucleus and, starting from the hard nucleus, the softer, more flexible areas of the lens are pulled towards the aspiration handpiece.

A tissue strand with a thickness (or diameter) which is constant at least in sections and/or a thickness (or diameter) which increases at least in sections brings the advantage that in all longitudinal sections the tissue strand can be provided with good strength or a solid structure with respect to tearing. By diameter is meant not only a thickness of an at least approximately circular cross section but also a thickness of a cross-sectional geometry which deviates from the circular shape. The tissue strand preferably extends in all three spatial directions.

According to a variant, the control device can be set up to arrange the cutting path in such a way that the tissue strand has an at least approximately constant elasticity in relation to a longitudinal section of the tissue strand. The advantage of a constant elasticity in relation to a particular longitudinal section of the strand is that the strand is comparably stable in all longitudinal sections when the strand is aspirated and pulled starting from a point arranged centrally in the tissue, with the result that the danger of the strand tearing can be lessened. The elasticity of the lens tissue increases radially towards the outside. As the thickness or the diameter of the strand becomes larger at least in sections towards the outside, in particular continuously, a strand can be formed which also has an approximately constantly large cross-sectional area when the more elastic tissue is pinched by the suctioning-off.

An elasticity of the lens in different longitudinal sections can be estimated e.g. depending on the radial position of a respective section of the cutting path, in particular on the basis of the difference in elasticity usually present between a lens nucleus and a lens cortex.

According to an embodiment, the cutting path is formed by at least two different spiral-shaped curves in each case starting from a starting point arranged within the tissue. Here, at least two individual tissue strands can be provided, wherein each tissue strand can subdivide the tissue completely in the radial direction. The division into two individual tissue strands can be advantageous in respect of a movement of the focal point in particular because forming the respective cutting path along a two-dimensional curve can be sufficient.

The division into two separate tissue strands can also be advantageous if, before or after the tissue strand is cut, the lens is to be divided into two parts through a section plane. This can be expedient e.g. in the case of very hard lens nuclei. The starting point can be the same for both tissue strands.

According to an embodiment, the control device is set up to move the focal point to divide the tissue into at least two parts, in particular before a movement according to a continuous cutting path starting from a starting point arranged within the respective part of the tissue is effected in each of the parts.

The tissue can preferably be divided in at least one in particular centrally running section plane. Further preferably, a division is effected into several slices which preferably each have a thickness which corresponds at least approximately to an internal diameter of a lumen of an aspiration handpiece. Here, each slice can be divided easily into a spiral-shaped tissue strand which does not exceed a maximum thickness. This makes it easier to suction off the tissue strand.

A division of the lens can be expedient e.g. when the lens has a very hard nucleus. It can then be advantageous to work in two planes in the area of the nucleus. The requirement for a division depends on the thickness of the lens and the hardness of the lens as well as on the aspiration handpiece used.

A division of the lens is preferably effected in the horizontal direction, in particular in an equatorial region of the lens, into an upper and a lower half. However, the division can also be effected in such a way that a first part and a second part remain connected to each other, in particular at a point which corresponds to an end of the cutting path of the first part and a start of the cutting path of the second part, with the result that, even in the case of a division, a single, continuous lens strand is formed.

Preferably, the individual lens parts are also dissected into the shape of a spiral when the lens is divided, and are continuous and form a spiral chain.

In an embodiment, a laser system with a laser, in particular a femtosecond laser, and with a control device is provided, wherein the control device has at least one actuator and is set up to move, by means of the at least one actuator, a focal point of a laser beam of the laser according to the continuous cutting path predetermined by the control device.

In an embodiment, a method for controlling a laser system having a laser, in particular a femtosecond laser, is provided, wherein the laser system is set up to introduce a cut into tissue of a human or animal eye, in particular into a lens of the eye, in which the laser is controlled in such a way that, in particular depending on a distance of the focal point in relation to a reference point of the laser and/or on a position or geometry of the tissue, a focal point of a pulsed laser beam of the laser is moved along a continuous cutting path extending two- or three-dimensionally, in particular in a single cut, starting from a starting point arranged within the tissue, in particular in the whole tissue, between the starting point and an outer edge of the tissue and the tissue is dissected into at least one continuous, in particular spiral-shaped, tissue strand.

In the method, a distance of the focal point can be determined in relation to a reference point of the laser. Furthermore, a position and/or geometry of the tissue can be captured relative to the reference point and the focal point can be arranged within the tissue relative to the reference point. This makes it easier to move the focal point as geometrically precisely as possible in dependence on a particular tissue geometry. The cut or cutting path can, in particular, be arranged in a convex body, namely in the lens of the eye. The method can be developed as described in connection with the control device. The cutting path is preferably arranged at least in sections at a predeterminable minimum distance from an outer edge, in particular from a surface, of the tissue. Further embodiments of the method result from the embodiments described in connection with the control device.

The control method can be carried out in conjunction with the therapeutic procedure described below. Firstly, the laser introduces one or more incisions and carries out the subdivision of the lens according to an embodiment of the invention. The lens is dissected by means of the laser but is not necessarily cut up into several small pieces. The capsule membrane can then be removed using an aspiration handpiece. The lens itself can also be pulled out of the capsular bag using the same aspiration handpiece. It is hereby possible to dispense with an instrument change. Since an instrument change is not necessarily required, the aspiration handpiece can also have a somewhat larger diameter. The time required for the treatment can hereby be reduced, and/or the negative pressure for the suctioning-off can be reduced, which decreases the risk of the capsular bag being damaged. The treatment can also be carried out more easily as the aspiration handpiece does not have to be moved within the eye, or at most only minimally, in particular when it has a comparatively large diameter. The stress at the incision point and thus the post-operative astigmatism induced is reduced. An instrument change can at most be required when the nucleus of the lens is very hard and should be further emulsified for the purpose of an easier suctioning-off.

A free (distal) end of the aspiration handpiece is preferably positioned in a suction point arranged centrally in the lens. This reduces the risk of damaging the capsular bag. The lens is pulled away from the capsular bag. The hard (nucleus) areas of the lens are suctioned off first and only after that the areas arranged radially further out (lens cortex). This sequence increases the probability that the lens can be suctioned off completely in one strand. In this arrangement of the aspiration handpiece it is also possible to consider increasing the suction speed, in particular using a higher negative pressure, since a clear distance from the capsular bag can be maintained and the risk of damaging the capsular bag is significantly reduced. The risk of rupturing the capsule can be significantly reduced. The duration of the treatment can also be shortened hereby. The stress at the incision point, and thus the post-operative astigmatism induced, is reduced. The irrigation fluid rinsing volume, which is known to be directly related to the loss of endothelial cells during every cataract operation, can be massively reduced. In the ideal case, during the suctioning-off of the lens strand only as much irrigation fluid is fed into the eye as is lost through the volume of the suctioned-off lens.

Thus, owing to the arrangement of the cutting path according to an embodiment of the invention, both a dissection starting from a centrally arranged starting point and a suctioning-off in a central point can be effected. In both method steps, the risk of damaging the capsular bag is significantly less than in conventional methods in which the lens is also dissected in the edge area and individual parts of the lens remain directly next to the capsular bag and have to be suctioned off there too. In contrast, with a lens dissected into a lens strand, an operator can arrange the free end of the aspiration handpiece centrally and leave it in this central position. The lens can be suctioned off completely without the aspiration handpiece having to be shifted or having to be moved close to the capsular bag.

In an embodiment, a computer program product with code is provided, which is set up to carry out a method according to an embodiment of the invention on a computer or an arithmetic unit. In another embodiment, a memory in which the computer program product is stored is provided.

In an embodiment, a method for introducing a cut into tissue of a human or animal eye, in particular into a lens of the eye, by means of a laser, in particular a femtosecond laser, with the steps of:
1. determining a distance of a focal point of a pulsed laser beam of the laser in relation to a reference point of the laser;
2. capturing a position and/or geometry of the tissue relative to the reference point and arranging the focal point at a starting point within the tissue; and
3. moving the focal point in such a way in the tissue, in particular in dependence on the distance and on the position and/or geometry, that a cut is made in the tissue according to a continuous cutting path extending two- or three-dimensionally starting from the starting point and the tissue is dissected, in particular by means of a single cut, into at least one in particular spiral-shaped tissue strand between the starting point and an outer edge of the tissue.
   The laser radiation is introduced into the tissue starting from an inner, preferably centrally arranged starting point, wherein the focal point can dissect the entire tissue (the entire lens) by means of geometrically matched guidance of the focal point. At the edge or in the area of the surface, the focal point can be kept at a particular distance from the edge or the surface in order to avoid damage to the capsular bag. The entire tissue can hereby be dissected into one strand, which can be suctioned off from the inside without damaging the capsular bag, in particular in one pull.

In an embodiment, a method is provided for removing tissue, in particular a lens, from a human or animal eye, with the steps of:
1. ubdividing the tissue, in particular the entire tissue, by means of a laser, in particular a femtosecond laser, in such a way that starting from a starting point arranged within the tissue the tissue forms at least one in particular spiral-shaped tissue strand between the starting point and an outer edge of the tissue;
2. positioning a free end of an aspiration handpiece in the starting point, in particular centrally within the tissue; and
3. suctioning off the tissue by aspirating the at least one tissue strand starting from the starting point, in particular in the case of a free end of the aspiration handpiece arranged statically at the starting point.
   With this method, the advantages which were already mentioned result. In the case of a static arrangement of the aspiration handpiece centrally within the tissue, the risk of damaging the capsular bag can be decreased. The stress at the incision point and thus the post-operative astigmatism induced is reduced. The irrigation fluid rinsing volume, which is known to be directly related to the loss of endothelial cells during every cataract operation, can be massively reduced. In the ideal case, during the suctioning-off of the lens strand only as much irrigation fluid is fed into the eye as is lost through the volume of the suctioned-off lens. The cutting path is preferably arranged in sections at a predeterminable minimum distance from the outer edge, in particular from a surface, of the tissue.

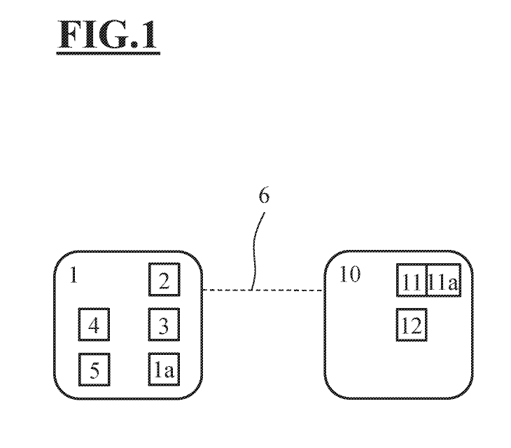

In FIG. 1 a control device 1 is shown which has a focus-measuring apparatus 2, an optical capture unit 3, an arithmetic unit 4 as well as a memory 6. The control device 1 has one or more actuators 1a and is connected to a laser system 10 via a communication interface 6. The communication interface 6 can be designed wireless and/or wired. The laser system 10 has a femtosecond laser 11 with an optics apparatus 11a as well as one or more actuators 12. The control device 1 is set up to control the actuator or actuators 1a, 12 in such a way that a focal point of a laser beam of the femtosecond laser 11 is guided on a continuous, spiral-shaped cutting path.

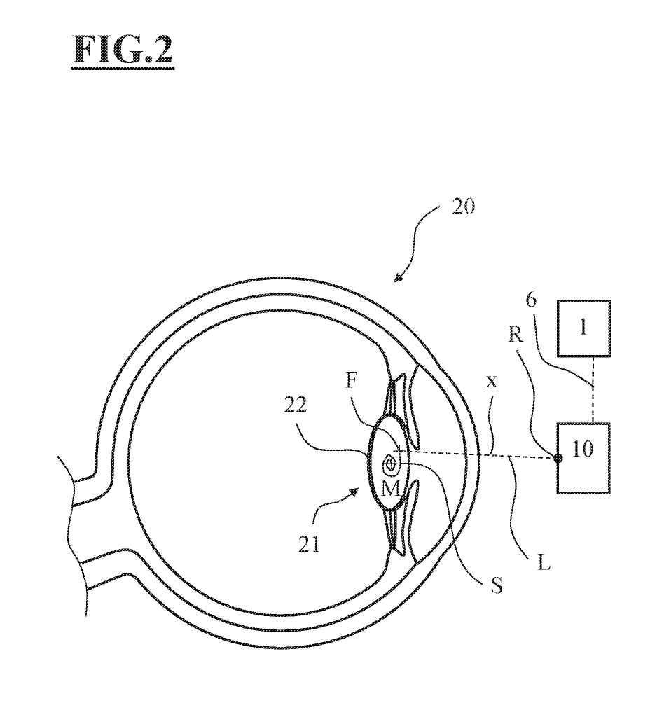

In FIG. 2, a human eye 20 is shown which has a lens 21 with a central point M. The lens 21 is arranged in a capsular bag 22 which holds the lens 21 in position and which also serves to receive an artificial lens (not represented) which replaces the lens 21. The lens 21 is irradiated by a laser beam L of a laser system 10 which is bundled in a focal point F which is moved continuously within the lens 21 and introduces a cut along a cutting path S into the lens 21. The laser system 10 is in communication via a connection 6 with a control device 1 which controls the laser system 10 in such a way that the focal point F is moved along the cutting path S which is predetermined or which is calculated by the control device 1. The focal point F is arranged at a distance x from a reference point R of the laser system 10 or a laser of the laser system 10.

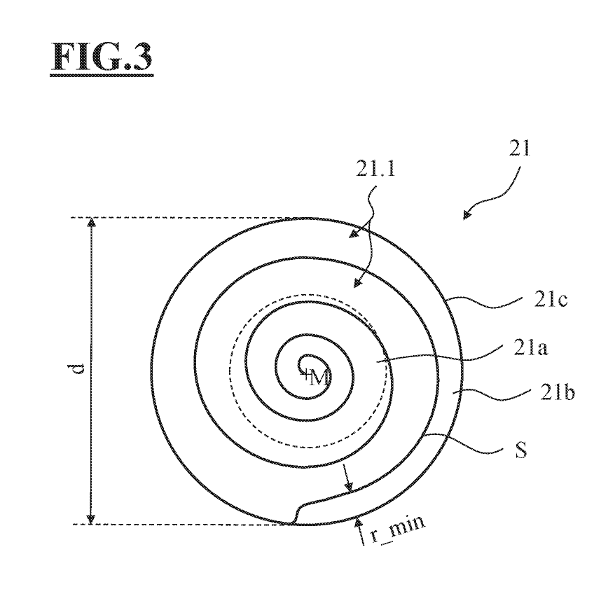

In FIG. 3, a lens 21 is shown which is dissected by a spiral-shaped cutting path S, wherein the cutting path S extends radially outwards in a spiral starting from a central point M of the lens 21 and goes through both a nucleus 21a of the lens 21 and a cortex 21b of the lens 21. The lens 21 is dissected into a single, continuous lens strand 21.1. The lens 21 has a diameter d and the cutting path S extends in all areas of the diameter of the lens 21. The cutting path S is guided to an edge or a surface 21c of the lens 21, wherein the cutting path S maintains a minimum distance r_min from the surface 21c. Alternatively, the cutting path can also end at a particular minimum distance r_min from the surface 21c. The cutting path S is represented by way of example and greatly simplified, wherein the distances and geometries of the cutting path S which are preferably to be maintained can differ from the representation. In particular, the cutting path can also assume a three-dimensional geometry.

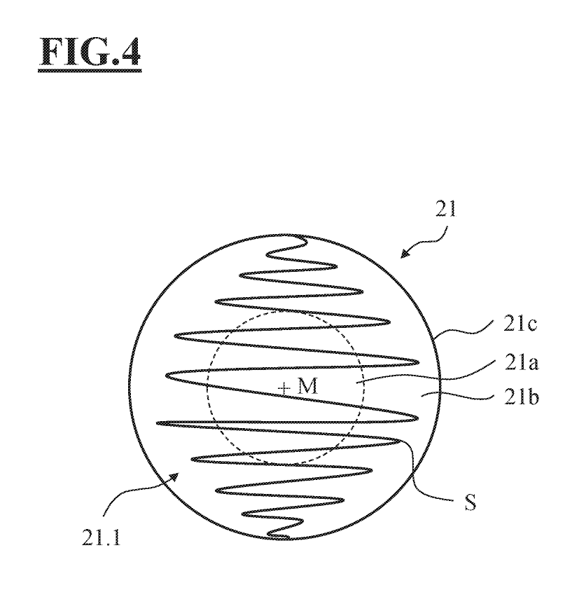

In FIG. 4, a lens 21 is shown which is dissected by a spiral-shaped cutting path S, wherein the cutting path S extends radially outwards, and both upwards and downwards, in a spiral starting from a central point M of the lens 21 and goes through both a nucleus 21a of the lens 21 and a cortex 21b of the lens 21. The lens 21 is dissected into a continuous lens strand 21.1. The cutting path S is guided to a surface 21c of the lens 21. Alternatively, the cutting path can also end at a specific minimum distance from the surface 21c. The cutting path S is represented by way of example and greatly simplified, wherein the distances and geometries of the cutting path S which are preferably to be maintained can differ from the representation. In particular, the cutting path can also assume a three-dimensional geometry.

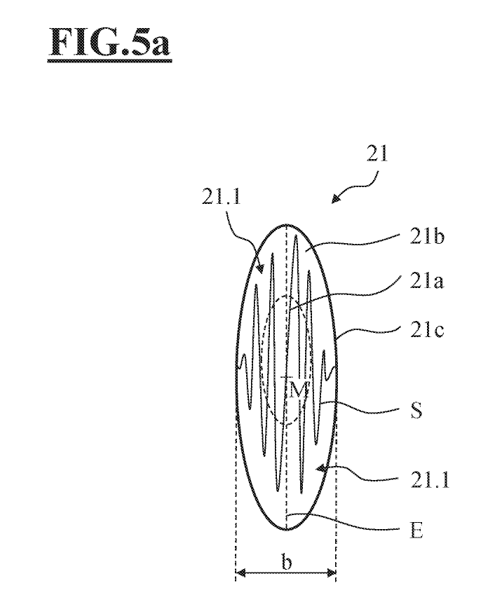

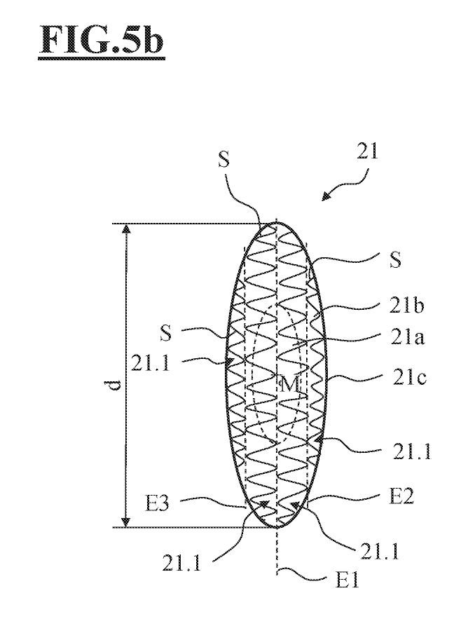

In FIG. 5a, a lens 21 is shown which is dissected by a spiral-shaped cutting path S, wherein the cutting path S extends radially outwards, and both towards the right and towards the left, in a spiral starting from a central point M of the lens 21 and goes through both a nucleus 21a of the lens 21 and a cortex 21b of the lens 21. The lens 21 has a thickness b and the cutting path S extends along the entire thickness of the lens 21. The lens 21 is dissected in a vertically arranged dissection plane E (in particular a plane with a maximum radial extent of the lens geometry) into two areas of comparable size which do not necessarily have to be completely separated from each other, but can be connected to each other. The lens 21 is dissected into one or two in each case continuous lens strands 21.1. The cutting path S is guided to a surface 21c of the lens 21. Alternatively, the cutting path can also end at a particular minimum distance from the surface 21c. The cutting path S is represented by way of example and greatly simplified, wherein the distances and geometries of the cutting path S which are preferably to be maintained can differ from the representation. In particular, the cutting path can also assume a three-dimensional geometry.

In FIG. 5b, the lens 21, which is dissected by one or more spiral-shaped cutting paths S, is shown divided into four slices in several dissection planes E1, E2 and E3, wherein the (respective) cutting path S extends radially outwards in a spiral starting from a central point M of the lens 21. The lens 21 has a diameter d and the cutting path S extends along the entire diameter. The four slices can have a comparable thickness, with the result that a (respective) tissue strand can be formed with a comparable thickness, in particular matching the internal diameter of a lumen of an aspiration handpiece.

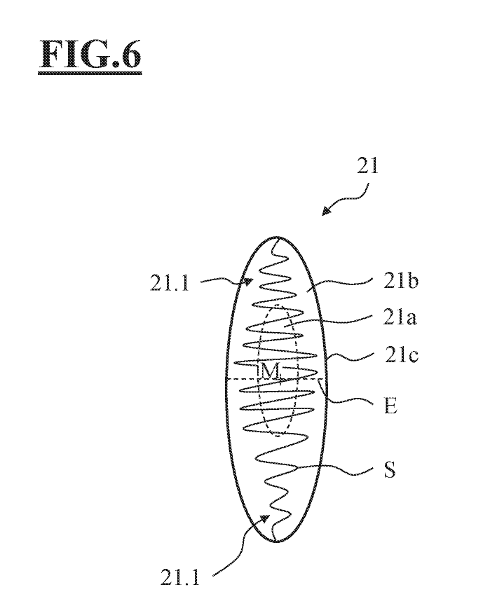

In FIG. 6, a lens 21 is shown which is dissected by a spiral-shaped cutting path S, wherein the cutting path S extends radially outwards, and both upwards and downwards, in a spiral starting from a central point M of the lens 21 and goes through both a nucleus 21a of the lens 21 and a cortex 21b of the lens 21. The lens 21 is dissected in a horizontally arranged dissection plane E (in particular a plane with a maximum thickness of the lens geometry) into two areas of comparable size which do not necessarily have to be completely separated from each other, but can be connected to each other. The lens 21 is dissected into one or two in each case continuous lens strands 21.1. The cutting path S is guided to a surface 21c of the lens 21. Alternatively, the cutting path can also end at a particular minimum distance from the surface 21c. The cutting path S is represented by way of example and greatly simplified, wherein the distances and geometries of the cutting path S which are preferably to be maintained can differ from the representation. In particular, the cutting path can also assume a three-dimensional geometry.

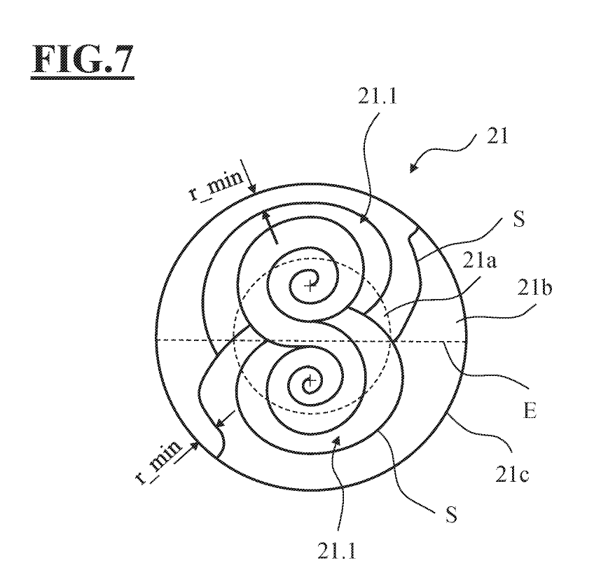

In FIG. 7, a lens 21 is shown which is dissected by two spiral-shaped cutting paths S, wherein the cutting paths S extend radially outwards in a spiral starting from two points arranged within the lens 21 and go through both a nucleus 21a of the lens 21 and a cortex 21b of the lens 21. The lens 21 is dissected in a horizontally arranged dissection plane E (in particular a plane with a maximum thickness of the lens geometry) into two areas of comparable size which do not necessarily have to be completely separated from each other, but can be connected to each other. The lens 21 is dissected into one or two in each case continuous lens strands 21.1. The cutting paths S are in each case guided to an edge or a surface 21c of the lens 21. The cutting paths S in each case maintain a minimum distance r_min from the surface 21c. Alternatively, the cutting paths S can also end at a particular minimum distance from the surface 21c. The cutting paths S are represented by way of example and greatly simplified, wherein the distances and geometries of the cutting paths S which are preferably to be maintained can differ from the representation. In particular, the cutting paths can also assume a three-dimensional geometry.

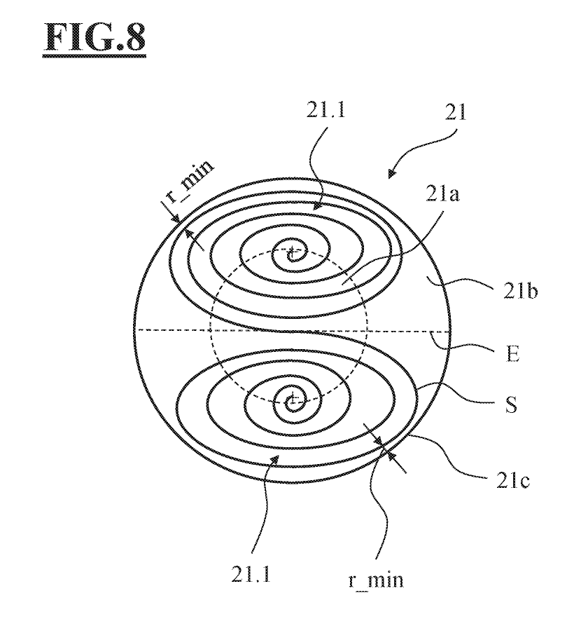

In FIG. 8, a lens 21 is shown which is dissected by a spiral-shaped cutting path S, wherein the cutting path S extends radially outwards in a spiral starting from two points arranged within the lens 21 and goes through both a nucleus 21a of the lens 21 and a cortex 21b of the lens 21. The lens 21 is dissected in a horizontally arranged dissection plane E (in particular a plane with a maximum thickness of the lens geometry) into two areas of comparable size which are connected to each other via the cutting path S. The lens 21 is dissected into one or two in each case continuous lens strands 21.1. The cutting path S is guided at a minimum distance r_min from a surface 21c of the lens 21 and does not run to the surface 21c. The cutting path S is represented by way of example and greatly simplified, wherein the distances and geometries of the cutting path S which are preferably to be maintained can differ from the representation. In particular, the cutting path can also assume a three-dimensional geometry. In the case of a continuous cutting path starting from a starting point arranged within the lens, in particular in the central point of the lens, between the starting point and an outer edge of the tissue the lens can be dissected such that it can be suctioned off in one piece without there being the risk of damaging the capsular bag.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE NUMBERS

1 Control device
1a Actuator
2 Focus-measuring apparatus
3 Optical capture unit
4 Arithmetic unit
5 Memory
6 Connection between control device and laser system
10 Laser system
11 Laser, in particular femtosecond laser
11a Optics apparatus
12 Actuator
20 Eye
21 Lens
21a Lens nucleus
21b Lens cortex
21c Surface or edge
21.1 Lens strand
22 Capsular bag
b Thickness of the lens
d Diameter of the lens
r_min Minimum distance of cutting path from edge or from surface
x Distance of focal point from reference point
E, E1, E2, E3 Dissection planes
F Focal point
M Central point or centrally arranged point of the lens
L Laser beam
R Reference point
S Cutting path

1. A control device for a laser system with a laser for introducing a cut into tissue of a human or animal eye, wherein the control device is set up to arrange and move a focal point of a laser beam of the laser within the tissue, the control device comprising:
an actuator configured to move the focal point along a continuous cutting path starting from a starting point arranged within the tissue between the starting point and an outer edge of the tissue in order to dissect the tissue into at least one continuous tissue strand.

2. The control device according to claim 1, wherein the control device is configured to capture a position of the outer edge of the tissue and to arrange the cutting path at least in sections at a predeterminable minimum distance from the outer edge.

3. The control device according to claim 1, wherein the control device is configured to capture a central point of the tissue and to arrange the cutting path around the central point.

4. The control device according to claim 1, wherein the control device is configured to move the focal point in order to form the tissue strand with a constant thickness and/or a thickness which increases at least in sections.

5. The control device according to claim 1, wherein the cutting path is formed by at least two different spiral-shaped curves, each starting from a starting point arranged within the tissue.

6. The control device according to claim 1, wherein the control device is configured to move the focal point in order to divide the tissue into at least two parts.

7. A laser system, comprising:
a laser configured to produce a laser beam; and
a control device having at least one actuator configured to move a focal point of the laser beam of the laser according to a continuous cutting path predetermined by the control device.

8. A method for controlling a laser system having a laser configured to introduce a cut into tissue of a human or animal eye, the method comprising:
controlling the laser such that a focal point of a pulsed laser beam of the laser is moved along a continuous cutting path starting from a starting point arranged within the tissue between the starting point and an outer edge of the tissue in order to dissect the tissue into at least one continuous tissue strand.

9. A computer program comprising code having instructions for controlling a laser configured to introduce a cut into tissue of a human or animal eye, the instructions comprising instructions for:
controlling the laser such that a focal point of a pulsed laser beam of the laser is moved along a continuous cutting path starting from a starting point arranged within the tissue between the starting point and an outer edge of the tissue in order to dissect the tissue into at least one continuous tissue strand.

10. A computer readable memory having stored thereon instructions for controlling a laser configured to introduce a cut into tissue of a human or animal eye, the instructions comprising instructions for:
controlling the laser such that a focal point of a pulsed laser beam of the laser is moved along a continuous cutting path starting from a starting point arranged within the tissue between the starting point and an outer edge of the tissue in order to dissect the tissue into at least one continuous tissue strand.
